SEQUENCE LISTING (7 sequences)

SEQ ID NO: 1 - DNA, 24 bp, Artificial Sequence
  Primer
gttggctctgcagggtctctagga

SEQ ID NO: 2 - DNA, 27 bp, Artificial Sequence
  Primer
cccaaacccccgtactggccagcaccc

SEQ ID NO: 3 - DNA, 15 bp, Artificial Sequence
  Primer
cggtctcctctggca

SEQ ID NO: 4 - DNA, 18 bp, Artificial Sequence
  Primer
tcaccgagctcccatctt

SEQ ID NO: 5 - DNA, 19 bp, Artificial Sequence
  Primer
ggggcaggtgtgtcttcag

SEQ ID NO: 6 - DNA, 21 bp, Artificial Sequence
  Primer
atggacttccacattagggac

SEQ ID NO: 7 - DNA, 22 bp, Artificial Sequence
  Primer
gaaggtagtggattctccatca

(19) United States
(12) Patent Application Publication
(10) Pub. No.: US 2007/0134675 A1
(43) Pub. Date: Jun. 14, 2007

(54) Alterations in the copy number of the SULT1A1 gene

(76) Inventors: Scott J. Hebbring, Rochester, MN (US); Stephen Thibodeau, Rochester, MN (US); Araba A. Adjei, Rochester, MN (US); Richard M. Weinshilboum, Rochester, MN (US)

Correspondence Address: FISH & RICHARDSON P.C., PO BOX 1022, MINNEAPOLIS, MN, 55440-1022, US

(21) Appl. No.: 11/297,712

(22) Filed: Dec. 8, 2005

(51) Int. Cl. C12Q 1/68; C12M 1/34

(52) U.S. Cl. 435/6; 435/287.2

(57) ABSTRACT

Methods are described for determining sulfonator status of a patient and determining dosages of drugs based on copy number of the SULT1A1 gene.

### STATEMENT AS TO FEDERALLY SPONSORED RESEARCH

Funding for the work described herein was provided in part by the federal government under Grant Nos. GM28157, GM35720, GM61388, CA72818, and CA91956. The federal government may have certain rights in the invention.

### TECHNICAL FIELD

This invention relates to methods and materials for determining copy number of a gene, and more particularly, to methods and materials for determining the copy number of the SULT1A1 gene, and assessing the sulfonator status of a subject based on SULT1A1 copy number.

### BACKGROUND

Cytosolic sulfotransferase superfamilies (SULTs) act on a wide variety of natural and synthetic chemicals. With the wide tissue distribution, extensive substrate affinity, involvement in the detoxification and metabolism of numerous drug compounds, hormones and xenobiotics, and the participation in the bioactivation of environmental and dietary procarcinogens, the SULT1A1 gene has been the most widely studied. Glatt, H. (2000) Chem. Biol. Interact. 129(1-2): 141-70.

SULT1A1 is one of four SULT1A genes located on chromosome 16 that share at least 93% amino acid sequence identity. Her et al. (1996) Genomics 33(3): 409-20; Raftogianis et al. (1996) Pharmacogenetics 6(6): 473-87; and Hildebrandt et al. (2004) Biochem. Biophys. Res. Commun. 321(4): 870-8. Three non-synonymous single nucleotide polymorphisms (SNPs) divide the SULT1A1 gene into four alleles: SULT1A1*1 (wildtype), SULT1A1*2 (Arg213His), SULT1A1*3 (Met223Val), and SULT1A1*4 (Arg37Gln). The two most common alleles found among most populations are the 1A1*1 and 1A1*2 alleles. Subjects homozygous for 1A1*2 are thought to have an 85% reduction in platelet phenol sulfotransferase activity and a decrease in thermal stability compared to subjects that are heterozygous for 1A1*1/1A1*2 or homozygous for 1A1*1. See Raftogianis et al. (1997) Biochem. Biophys. Res. Commun., 239(1):298-304; and Raftogianis et al. (1999) Biochem. Pharmacol., 58(4):605-16.

Langsenlehner et al (2004, Breast Cancer Res. Treat., 87(1):19-22) showed that in their breast cancer population, the 1A1*2 allele was associated with lymph node metastasis but not with breast cancer itself. Saintot et al (2003, Int. J. Cancer, 107(4):652-7) also found no association of the 1A1*2 allele to breast cancer in the population they studied; however they observed that the 1A1*2 allele increased the risk for breast cancer if one smoked. Within a Chinese population, the frequency of the 1A1*2 allele in women with breast cancer was statistically significant compared to a control set. Han et al. (2004) Toxicol. Lett., 150(2):167-77. Conversely, in other studies, the 1A1*1 allele may be associated with prostate and bladder cancer. Nowell et al. (2004) Cancer Epidemiol. Biomarkers Prev., 13(2):270-6; and Zheng et al. (2003) Cancer Lett., 202(1):61-9. Based on these variable results, other factors may play a critical role in SULT1A1's function and involvement with certain types of cancers.

### SUMMARY

The invention is based on the discovery that the copy number of the SULT1A1 gene is altered in at least 30% of the human subjects studied. Deletions or duplications of the SULT1A1 gene can result in alteration in SULT1A1 activity, and consequently, the administration of improper dosages of drugs to patients or an increased risk of cancer as SULT1A1 is involved in the detoxification and metabolism of numerous drugs, hormones, and xenobiotics, and participates in the bioactivation of environmental and dietary procarcinogens.

In one aspect, the invention features a method of determining sulfonator status of a patient. The method includes providing a biological sample from the patient; determining copy number of the SULT1A1 gene in the biological sample (e.g., a blood or tissue sample); and correlating copy number of the SULT1A1 gene with sulfonator status of the patient. Copy number can be determined by analyzing DNA, RNA or protein. For example, copy number of the SULT1A1 gene can be detected by a quantitative PCR assay such as a fluorescent quantitative PCR assay, fluorescence in situ hybridization, Southern blotting, multiplex ligation-dependent probe amplification (MLPA), or Quantitative Multiplex PCR of Short Fluorescent Fragments (QMPSF). Copy number also can be detected by Northern blotting or Western blotting.

In another aspect, the invention features a method for determining the dosage of a drug to be administered to a patient, wherein the drug is a substrate of SULT1A1. The method includes providing a biological sample (e.g., blood or tissue sample) from the patient; determining copy number of the SULT1A1 gene in the biological sample; and determining the dosage of the drug based, at least in part, on the copy number of the SULT1A1 gene. An increase in the copy number of the SULT1A1 gene can result in an increased dosage of the drug, whereas a decrease in copy number of the SULT1A1 gene can result in a decreased dosage of the drug. Copy number of the SULT1A1 gene can be determined by a fluorescent quantitative PCR assay, fluorescence in situ hybridization, Southern blotting, MLPA, or QMPSF. The drug can be a monocyclic phenol, epinephrine, acetaminophen, or minoxidil.

The invention also features an article of manufacture that includes a first oligonucleotide primer and a second oligonucleotide primer, wherein the first and second primers, in the presence of mammalian genomic DNA and under polymerase chain reaction conditions, produce a first nucleic acid product corresponding to a region of a SULT1A1 gene and a second nucleic acid product corresponding to a non-polymorphic region of a mammalian genome, and wherein the first and second nucleic acid products are different lengths. The non-polymorphic region of a mammalian genome can be the SULT1A2 gene or the SULT1A3/SULT1A4 gene. The article of manufacture further can include a third oligonucleotide primer and a fourth oligonucleotide primer, wherein the third and fourth primers, in the presence of mammalian genomic DNA and under polymerase chain reaction conditions, produce a third nucleic acid product corresponding to a region of a control gene (e.g., the coagulation factor five gene or GAPDH gene). The first or second primer can be labeled (e.g., with a fluorescent dye or a radioisotope).

In yet another aspect, the invention features a method of determining copy number of a gene. The method includes providing a biological sample containing mammalian genomic DNA; producing first and second nucleic acid products from the biological sample using a first oligonucleotide primer and a second oligonucleotide primer under polymerase chain conditions, the first nucleic acid product corresponding to a region of a target gene and the second nucleic acid product corresponding to a non-polymorphic region of a mammalian genome, and wherein the first and second nucleic acid products are different lengths, and determining copy number based on the relative proportion of the first and second nucleic acid products. The target gene can be the human SULT1A gene. The non-polymorphic region of the mammalian gene can be the human SULT1A2 or SULT1A3/SULT1A4 gene.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description and drawings, and from the claims.

### DESCRIPTION OF DRAWINGS

FIG. 1 is a graph of the ratio of SULT1A1/SULT1A2 copy number (diamonds) and corresponding SULT1A1 activity (squares).

### DETAILED DESCRIPTION

In general, the invention provides methods and materials for determining sulfonator status of a mammal (e.g., a human) based on the copy number of the SULT1A1 gene. “Sulfonator status” refers to the ability to transfer a sulfate group to a substrate. In particular, SULT1A1 catalyzes the transfer of inorganic sulfate to molecules such as dopamine, epinephrine, acetaminophen, 17β-estradiol (E2), diethylstilbestrol, 1-napthol, 4-hydroxytamoxifen, minoxidil, estrone (E1), genistein, catechin hydrate, epicatechin, epigallocatechin gallate, quercetin, myricetin, kaempferol, caffeic acid chlorgenic acid, n-propyl gallate, resveratrol, nitrophenol, and other monocyclic phenols, and uses 3′-phosphoadenosine-5′-phosphosulfate (PAPS) as the sulfate donor. Sulfonation typically detoxifies compounds, as the resulting ionized, organic sulfates are more readily excreted than the unsulfated compounds. Furthermore, functional groups that may interact with biological macromolecules such as nucleic acids or proteins can be masked by the sulfate moiety. Certain substrates, however, become more reactive upon sulfonation. For example, the N-hydroxy metabolite of 2-acetylaminoflourene is converted to a N—O-sulfate ester, which is reactive with biological macromolecules.

Thus, determining copy number of the SULT1A1 gene can facilitate the prediction of therapeutic efficacy and toxicity of drugs on an individual basis since individuals carrying 3 or more copies of the SULT1A1 gene can be greater metabolizers of numerous drugs, and as such, have inadequate therapeutic responses due to a higher metabolism rate. Conversely, individuals with <2 copies of the SULT1A1 gene can be at risk for toxicity due to decreased metabolism of drugs. In addition, alterations in SULT1A1 copy number may play a role in cancers, including, for example, breast cancer (and increased breast density after oral estrogen, a risk factor for breast cancer), colon cancer, esophageal, and lung cancer.

Determining Copy Number of a Gene

SULT1A1 chromosomal copy number can be detected by any DNA, RNA (e.g., Northern blotting), or protein (e.g., Western blotting or protein activity) based method. Non-limiting examples of DNA based methods include quantitative PCR; fluorescence in situ hybridization (FISH); Southern blotting; multiple amplifiable probe hybridization (MAPF, see Hollox et al., 2002, Expert Rev. Mol. Diagn., 2(4):370-8.); multiplex ligation-dependent probe amplification (MLPA, see Schouten et al., 2002, Nucleic Acids Res., 30(12):e57, kits available from MRC-Holland, Amsterdam, The Netherlands); QMPSF (Quantitative Multiplex PCR of Short Fluorescent Fragments, see Casilli et al., 2002, Hum. Mutat. 20(3):218-26), and combinations of such methods.

Typically, genomic DNA is used in the analysis of copy number. Genomic DNA can be extracted from any biological sample containing nucleated cells, such as a peripheral blood sample or a tissue sample (e.g., mucosal scrapings of the lining of the mouth or from renal or hepatic tissue). Standard methods can be used to extract genomic DNA from a blood or tissue sample, including, for example, phenol extraction. Alternatively, genomic DNA can be extracted with kits such as the QIAamp® Tissue Kit (Qiagen, Valencia, Calif.), Wizard® Genomic DNA purification kit (Promega, Madison, Wis.) and the A.S.A.P.™ Genomic DNA isolation kit (Boehringer Mannheim, Indianapolis, Ind.).

Quantitative PCR assays can include quantitative-fluorescent PCR (QF-PCR) assays and real-time quantitative PCR assays such as the TaqMan® assay (Applied Biosystems, Foster City, Calif.) or the LightCycler® assay (Roche Diagnostics Corporation, Indianapolis, Ind.). QF-PCR generally involves the amplification of genomic DNA using a pair of primers. Typically, one of the primers (e.g., the forward primer) is labeled with a fluorescent molecule to allow the size and amount of the amplified PCR product to be assessed.

As described herein, quantitative PCR also can be performed using a pair of oligonucleotide primers that co-amplify two fragments, a fragment from the gene of interest and a copy number control fragment from a non-polymorphic region of DNA. The amplified fragments differ in length from each other by at least 1 nucleotide (e.g., 2, 3, 4, 5, 6, 7, 8, 9, 10, 15, or more nucleotides), and can be 30, 40, 50, 60, 70, 80 90, 100, 125, 150, 175, 200, 225, 250, 300, 350, 400, or more nucleotides in length. Oligonucleotide primer pairs can amplify, for example, one or more exons, portions of exons, or introns or other non-coding sequences in the target sequences. For example, a pair of primers can amplify a region from the human SULT1A1 gene and a region from a different human SULT1A1 gene such as the SULT1A2 or SULT1A3/SULT1A4 gene. SULT 1A3 is duplicated on human chromosome 16 and is referred to as SULT1A3/SULT1A4 herein. See, Hildebrandt et al. (2004 ) Biochem. Biophys. Res. Commun. 321(4):870-8. The genomic and coding sequences of the human SULT1A1 gene are set forth in GenBank Accession Nos. U52852 and AB062428, respectfully. The genomic and coding sequences of the human SULT1A2 gene are set forth in GenBank Accession Nos. U28170 and NM_{—}177528, respectfully; and genomic and coding sequences of the human SULT1A3 gene are set forth in GenBank Accession Nos. U20499 and NM_{—}003166, respectfully. The oligonucleotide primers having the nucleotide sequences set forth in SEQ ID NO:4 and SEQ ID NO:5 are examples of primers that co-amplify fragments from the SULT1A1 (a 212 nucleotide fragment) and SULT1A2 (a 208 nucleotide fragment) genes.

In some embodiments, a quantitative PCR reaction can include one or more additional oligonucleotide primer pairs. For example, an oligonucleotide primer pair can be included such that an extra copy number control fragment is amplified. Suitable control fragments are from a non-polymorphic region of a mammalian genome (e.g., human genome) and include, for example, a region from the coagulation factor five or the glyceraldehyde-3-phosphate dehydrogenase (GAPDH) gene.

Typically, oligonucleotide primers are 10 to 50 nucleotides in length and can be combined with genomic DNA from a mammal and subjected to PCR conditions as set out below, to co-amplify a product that corresponds to a region of a gene of interest (e.g., the SULT1A1 gene) and a non-polymorphic region of a mammalian genome (e.g., a SULT1A2, or SULT1A3/SULT1A4 gene). Specific PCR conditions typically are defined by the concentration of salts (e.g., MgCl₂) in the reaction buffer, and by the temperatures utilized for melting, annealing, and extension. Specific concentrations or amounts of primers, templates, deoxynucleotides (dNTPs), and DNA polymerase also may be set out. For example, PCR conditions with a buffer containing 2.0 mM MgCl₂, and melting, annealing, and extension temperatures of 94° C., 55° C.-64° C. (e.g., 58° C.), and 72° C., respectively, are particularly useful. Under such conditions, a PCR sample can include, for example, 15 ng genomic DNA, 6 μM of each primer, 200 μM dNTPs, 1 U DNA polymerase (e.g., AmpliTaq Gold from Applied Biosystems), and the appropriate amount of buffer as specified by the manufacturer of the polymerase (e.g., 1×AmpliTaq Gold buffer). Denaturation, annealing, and extension each may be carried out for 30 seconds per cycle, with a total of 20 to 40 cycles (e.g., 23 cycles). An initial denaturation step (e.g., 94° C. for 2-10 minutes) and a final elongation step (e.g., 72° C. for 10 minutes) also may be useful.

Typically, one primer of each primer pair is labeled such that the amplified product can be detected. In embodiments in which multiple pairs of primers are used, each primer pair can be labeled with a different moiety such that multiple amplifications can be carried out in the same reaction. Suitable labels, include, for example, radioisotopes (e.g., ¹²⁵I, ¹³¹I, ³⁵S, ³H, ³²P, ³³P, or ¹⁴C), fluorescent moieties (e.g., fluorescein, carboxyfluorescein, fluorescein-5-isothiocyanate (FITC), PerCP, rhodamine, or phycoerythrin (PE)), luminescent moieties (e.g., Qdot™ nanoparticles supplied by the Quantum Dot Corporation, Palo Alto, Calif.), or compounds that absorb light of a defined wavelength. Methods of detecting or quantifying a label depend on the nature of the label and are known in the art. Examples of detectors include, without limitation, x-ray film, radioactivity counters, scintillation counters, spectrophotometers, colorimeters, fluorometers, luminometers, and densitometers.

Amplified products can be separated based on size (e.g., in a slab-gel system or by capillary electrophoresis) and the appropriate detection system used to determine the relative proportion of the amount of amplified product from the gene of interest to the amount of amplified product from the copy number control(s). In particular, an automated system for separating and detecting the amplified products, such as the ABI PRISM® 3100 capillary electrophoresis system from Applied Biosystems, can be used to separate the PCR products and determine the height ratio of the different amplified products to discriminate the copy number of the gene of interest. An approximate 1:1 ratio of the amount of product from the gene of interest to the amount of product from the copy number control indicates that copy number is normal (i.e., copy number is 2). An increase in the ratio of the amount of product from the gene of interest to the amount of product from the copy number control indicates that the copy number is increased (i.e., 3 or more copies), whereas a decrease in the ratio indicates that the copy number is decreased (i.e., 1 copy).

If the copy number control is a duplicated gene (i.e., 4 copies), the amount of product from the gene of interest to the amount of product from the copy number control can be performed as described above and the copy number adjusted accordingly. For example, a 1:1 ratio of the amount of product from the gene of interest to the amount of product from the copy number control of a duplicated gene indicates that the copy number of the gene of interest also is 4. An increase in the ratio of the amount of product from the gene of interest to the amount of product from the copy number control indicates that the copy number is increased (i.e., 5 or more copies). A decrease in the ratio can indicate that the copy number is normal or decreased, depending on the amount of product from the gene of interest. For example, a 1:2 ratio of the amount of product from the gene of interest to the amount of product from the copy number control indicates that copy number is normal and a 1:4 ratio indicates that copy number is decreased.

Assessing Sulfonator Status and Determining Dosages of Drugs

SULT1A1 copy number can be used to determine sulfonator status of a mammal or to determine the dosage of a drug to be administered to a patient. In general, methods of the invention include determining the copy number of the SULT1A1 gene in a biological sample from a patient (e.g., a human patient) relative to one or more regions of a control gene (e.g., the SULT1A2 gene and/or coagulation factor five gene). Methods for detecting copy number are described above.

In some embodiments, copy number can be correlated with sulfonator status. An increase in the copy number (e.g., 3, 4, 5, or 6 copies) of the SULT1A1 gene can result in an enhanced sulfonator status and an increased capacity for sulfonating substrates. A decrease in the copy number of the SULT1A1 gene can result in a decreased sulfonator status and a reduced capacity for sulfonating substrates.

Copy number of the SULT1A1 gene also can be used to determine the dosage of a drug-that is a substrate for SULT1A1, including, for example, estrogens such as estrone (E1), 17β-estradiol (E2), 2-hydroxyestrone, 2-hydroxyestradiol, 4-hydroxyestrone, and 4-hydroxyestradiol; synthetic estrogens such as diethylstilbestrol, 4-hydroxytamoxifen, and 2-methoxy estradiol; catecholamines and derivatives such as dopamine and tyramine; serotonin derivates such as 5-hydroxyindole and 6-hydroxymelatonin; drugs such as paracetamol, minoxidil, acetaminophen, and troglitazone; genistein, or other monocyclic phenols. An increase in copy number is indicative of an increased capacity for sulfonating substrates and as such, dosage of the drug may need to be increased to achieve an adequate therapeutic response. A decrease in copy number is indicative of a reduced capacity to sulfonate substrates and as such, dosage of the drug may need to be reduced to prevent toxicity. Additional factors to consider when determining the appropriate dosage of a drug can include the route of administration, the size, weight, surface area, age, and/or sex of the subject, or other drugs being administered. Dosages can be adjusted using standard empirical routines for optimization, as is well understood in the art.

Articles of Manufacture

Oligonucleotide primer pairs described herein can be combined with packaging materials and sold as articles of manufacture or kits for detecting copy number of a gene. Components and methods for producing articles of manufactures are well known. The articles of manufacture may combine one pair of oligonucleotide primers or a plurality of oligonucleotide primer pairs (e.g., 2, 3, 4, or more than 4 primer pairs). For example, an article of manufacture can include first and second oligonucleotide primers, which, in the presence of mammalian genomic DNA and under polymerase chain reaction conditions, produce a first nucleic acid product corresponding to a region of a SULT1A1 gene and a second nucleic acid product corresponding to a non-polymorphic region of a mammalian genome (e.g., a SULT1A2, or SULT1A3/SULT1A4 gene). In some embodiments, third and fourth oligonucleotide primers can be included in the article of manufacture that, in the presence of mammalian genomic DNA and under polymerase chain reaction conditions, produce a third nucleic acid product corresponding to a region of a control gene. Oligonucleotide primers can be labeled with a detectable moiety, for example, a fluorescent dye or radioisotope.

In addition, an article of manufacture further can include sterile water, pharmaceutical carriers, buffers, antibodies, indicator molecules, DNA polymerase, nucleotides, and/or other useful reagents for detecting copy number of a gene. Instructions describing how oligonucleotide primers can be used in an assay to detect copy number of a gene (e.g., SULT1A1) can be included in such kits.

The invention will be further described in the following examples, which do not limit the scope of the invention described in the claims.

### EXAMPLES

### Example 1

### Materials and Methods

DNA was obtained from the Coriell Cell Repository (Camden, N.J., U.S.A.). Specifically, 100 samples each from the two 100-item sample sets, HD100CAU and HD100AA, were used to perform these experiments. All of the DNA samples had been anonymized by the National Institutes of Health prior to their deposit in the Coriell Cell Repository, and all subjects had provided written consent for the use of their DNA for experimental purposes.

Total genomic DNA samples, which were previously isolated from 23 of 33 blood samples by Raftogianis et al. ((1997) Biochem. Biophys. Res. Cornmun. 239(1):298-304), also were used in this study. Platelet homogenates for these same samples had previously been prepared and phenotyped for SULT1A1 activity. See Van Loon and Weinshilboum (1984) Biochem. Genet. 22(11-12):997-1014; and Price et al. (1989) Genetics 122(4): 905-14.

Genotyping

Arg213His SNP genotyping was conducted using a PSQ 96 (Biotage, Uppsala, Sweden). PCR primers 5′/5BioTEG/GTT GGC TCT GCA GGG TCT CTA GGA3′ (SEQ ID NO:1) and 5′CCC AAA CCC CCG TAC TGG CCA GCA CCC3′ (SEQ ID NO:2) amplified a 333 bp fragment. Each 15 μL PCR reaction contained 15 ng lymphocyte DNA template, 0.6 μM of each primer, 200 μM dNTPs, 2.0 mM MgCl₂, and 1.0 U AmpliTaq Gold (Applied Biosystems). The PCR reaction consisted of a 10 minute incubation at 95° C., then 40 cycles of denaturation at 94° C. for 30 seconds, annealing at 63.5° C. for 30 seconds, and extension at 72° C. for 30 seconds, with a final extension at 72° C. for 10 minutes on a PTC-225 (MJ Research). The separation of double strand to single strand was conducted according to the Vacuum Prep Tool manual (Biotage). The sequencing reaction was conducted according to PSQ 96 protocol with sequencing primer 5′CGG TCT CCT CTG GCA3′ (SEQ ID NO:3).

Gene Duplication Assay: Fluorescent-Based Quantitative PCR

The set of PCR primers designed for this assay (5′6FAM/TCA CCG AGC TCC CAT CTT3′ (SEQ ID NO:4), located in exon 3, and 5′GGG GCA GGT GTG TCT TCAG3′ (SEQ ID NO:5), located in exon 4 of the SULT1A1 gene) co-amplify a 212 bp fragment within SULT1A1 and a 208 bp fragment within SULT1A2, which is used as a control copy number reference. In addition, a pair of primers (5′6FAM/ ATG GAC TTC CAC ATT AGG GAC3′ (SEQ ID NO:6) and 5′GAA GGT AGT GGA TTC TCC ATC A3′ (SEQ ID NO:7)) that amplify a 202 bp region from the Coagulation Factor Five gene was included as an additional copy number control. Each PCR reaction contained 15 ng template lymphocyte DNA, 0.6 μM of each primer, 200 μM dNTPs, 2.0 mM MgCl₂, and 1.0 U AmpliTaq Gold (Applied Biosystems) in a 15 μL reaction. The PCR reaction consisted of a 10 minute incubation at 95° C., then 23 cycles of denaturation at 94° C. for 30 seconds, annealing at 58° C. for 30 seconds, and extension at 72° C. for 30 seconds, with a final extension at 72° C. for 10 minutes on a PTC-225 (MJ Research).

Fragments were run on an ABI3100 (Applied Biosystems). By measuring the height ratio of the 212 bp amplicon of SULT1A1 to the reference 208 bp amplicon of SULT1A2, individuals were differentiated with one through >4 copies of SULT1A1.

### Example 2

### Duplication of SULT1A1

In genotyping-the SULT1A1 polymorphism Arg213His using a semi-quantitative sequencing assay (PSQ 96), it was observed that some of the Arg213His heterozygotes showed unusual peak distribution, suggesting that one allele was preferentially being amplified over the other. After confirming that there were no polymorphisms under the primer sequences and that SULT1A2, SULT1A3, or SULT1A4 were not being coamplified, it was hypothesized that these differences were caused by a polymorphic duplication of the gene.

A set of primers was designed that would co-amplify SULT1A1 and SULT1A2 equally but produce amplicons of different sizes. To confirm SULT1A2 also was not being duplicated or deleted, a secondary control amplicon (Factor 5) that was multiplexed with SULT1A1 and SULT1A2 was included in the assay. The same 100 Caucasian and 100 African-American samples that had been genotyped were tested using the fluorescent-based quantitative PCR assay described in Example 1. It was found that with the heterozygotes with unusual peak patterns, extra copies of the SULT1A1 gene were present. Not only were individuals identified with multiple copies of SULT1A1, two individuals were identified that were carriers of only one copy of SULT1A1. In the Caucasian population, 2.1% were carriers of one SULT1A1 copy and 22.1% had greater than two copies. In comparison, the African American population had no carriers of the gene deletion while 61.6% had greater than two copies (see Table 1). By looking at Arg213His in relation to copy number, it appears that SULT1A1*1 is the allele that is predominately, although not exclusively, duplicated. At least four of the samples carry duplicated SULT1A1*2 alleles. When comparing the pyrosequencing results to the fluorescent-based quantitative PCR assay results, virtually every sample that had uneven quantities of the SULT1A1*1 and 1A1*2 alleles had more than two alleles of SULT1A1.

TABLE 1

| | # of Alleles | | | | | |
|---|---|---|---|---|---|---|
| Samples | 1 | 2 | 3 | 4 | >4 | >2 |
| Caucasians (n = 95) | 2 (2.1%) | 72 | 18 | 3 (3.2%) | 0 | 21 (22.1%) |
| | | (75.8%) | (18.9%) | | | |
| African Americans | 0 | 38 | 37 | 21 | 3 (3.0%) | 61 (61.6%) |
| (n = 99) | | (38.4%) | (37.4%) | (21.2%) | | |

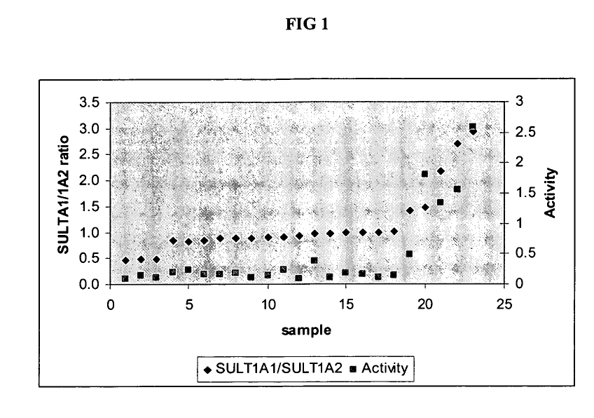

Within the 23 platelet samples with known SULT1A1 activity, three samples were identified carrying the deleted genotype while five samples had greater then two copies of the gene. These five samples showed an increase in SULT1A1 activity (FIG. 1) and the greater the number of alleles, the higher the activity (rho 0.8406, p<0.0001). This trend was not observed within the samples with only one allele. Because two allele activities are already low, the assay may not be sensitive enough to be able to pick up a further drop in activity caused by samples carrying only one allele.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of determining sulfonator status of a patient, said method comprising:
a) providing a biological sample from said patient;
b) determining copy number of the SULT1A1 gene in said biological sample; and
c) correlating copy number of the SULT1A1 gene with sulfonator status of said patient.

2. The method of claim 1, wherein said biological sample is a blood or tissue sample.

3. The method of claim 1, wherein copy number is determined by analyzing DNA, RNA or protein.

4. The method of claim 1, wherein copy number of the SULT1A1 gene is detected by a quantitative PCR assay.

5. The method of claim 4, wherein said quantitative PCR assay is a fluorescent quantitative PCR assay.

6. The method of claim 1, wherein copy number of the SULT1A1 gene is detected by fluorescence in situ hybridization.

7. The method of claim 1, wherein copy number of the SULT1A1 gene is detected by Southern blotting

8. The method of claim 1, wherein copy number of the SULT1A1 gene is detected by multiplex ligation-dependent probe amplification (MLPA).

9. The method of claim 1, wherein copy number of the SULT1A1 gene is detected by Quantitative Multiplex PCR of Short Fluorescent Fragments (QMPSF).

10. The method of claim 3, wherein copy number is detected by Northern blotting.

11. The method of claim 3, wherein copy number is detected by Western blotting.

12. A method for determining the dosage of a drug to be administered to a patient, wherein said drug is a substrate of SULT1A1, said method comprising:
a) providing a biological sample from said patient;
b) determining copy number of the SULT1A1 gene in said biological sample; and
c) determining the dosage of said drug based, at least in part, on the copy number of the SULT1A1 gene.

13. The method of claim 12, wherein said biological sample is a blood or tissue sample.

14. The method of claim 12, wherein said copy number of the SULT1A1 gene is detected by a fluorescent quantitative PCR assay.

15. The method of claim 12, wherein copy number of the SULT1A1 gene is detected by fluorescence in situ hybridization, Southern blotting, MLPA, or QMPSF.

16. The method of claim 12, wherein said drug is a monocyclic phenol.

17. The method of claim 12, wherein said drug is epinephrine, acetaminophen, or minoxidil.

18. An article of manufacture comprising a first oligonucleotide primer and a second oligonucleotide primer, wherein the first and second primers, in the presence of mammalian genomic DNA and under polymerase chain reaction conditions, produce a first nucleic acid product corresponding to a region of a SULT1A1 gene and a second nucleic acid product corresponding to a non-polymorphic region of a mammalian genome, and wherein said first and second nucleic acid products are different lengths.

19. The article of manufacture of claim 18, wherein said non-polymorphic region of a mammalian genome is the SULT1A2 gene.

20. The article of manufacture of claim 18, wherein said non-polymorphic region of a mammalian genome is the SULT1A3/SULT1A4 gene.

21. The article of manufacture of claim 18, said article of manufacture further comprising a third oligonucleotide primer and a fourth oligonucleotide primer, wherein said third and fourth primers, in the presence of mammalian genomic DNA and under polymerase chain reaction conditions, produce a third nucleic acid product corresponding to a region of a control gene.

22. The article of manufacture of claim 21, wherein said control gene is the coagulation factor five gene or GAPDH gene.

23. The article of manufacture of claim 18, wherein said first or second primer is labeled.

24. The article of manufacture of claim 23, wherein said first or second primer is labeled with a fluorescent dye or a radioisotope.

25. A method of determining copy number of a gene, said method comprising
a) providing a biological sample containing mammalian genomic DNA;
b) producing first and second nucleic acid products from said biological sample using a first oligonucleotide primer and a second oligonucleotide primer under polymerase chain conditions, said first nucleic acid product corresponding to a region of a target gene and said second nucleic acid product corresponding to a non-polymorphic region of a mammalian genome, and wherein said first and second nucleic acid products are different lengths; and
c) determining copy number based on the relative proportion of said first and second nucleic products.

26. The method of claim 25, wherein said target gene is the human SULT1A gene.

27. The method of claim 25, wherein said non-polymorphic region of said mammalian gene is the human SULT1A2 or SULT1A3/SULT1A4 gene.
